(19) United States
(12) Patent Application Publication
(10) Pub. No.: US 2025/0271355 A1
(43) Pub. Date: Aug. 28, 2025

(54) SURFACE ANALYSIS SYSTEM FOR AIR PURIFICATION UNIT AND AIR PURIFICATION UNIT INCLUDING THE SAME

(71) Applicants: Samsung Electronics Co., Ltd., Suwon-si (KR); Korea Institute of Energy Technology, Naju-si (KR)

(72) Inventors: Min Seok KOO, Suwon-si (KR); Wooyul KIM, Naju-si (KR); Cheolwoo PARK, Naju-si (KR); Gahye SHIN, Naju-si (KR); Hyun Chul LEE, Suwon-si (KR); Dongjin HAM, Suwon-si (KR)

(21) Appl. No.: 19/014,737

(22) Filed: Jan. 9, 2025

(30) Foreign Application Priority Data
Feb. 28, 2024 (KR) 10-2024-0029228

(51) Int. Cl. G01N 21/35; A61L 9/014; G01N 21/03; G01N 21/05

(52) U.S. Cl. CPC G01N 21/35; A61L 9/014; G01N 21/0332; G01N 21/05; A61L 2209/111; A61L 2209/14; G01N 2021/3595; G01N 2201/0233

(57) ABSTRACT

A surface analysis system for an air purification unit includes an air purification unit including a carrier and a metallic catalyst supported on the carrier, the air purification unit is configured to provide an air flow path in a first direction, a first light-source unit configured to irradiate inspection light onto a surface of the air purification unit, and an analysis unit configured to determine information regarding the surface of the air purification unit by receiving the inspection light reflected by the surface of the air purification unit, wherein the inspection light has an infrared wavelength, and the analysis unit is further configured to obtain oxidation number information of the metallic catalyst through infrared spectroscopy.

### CROSS-REFERENCE TO RELATED APPLICATION

This application claims priority to Korean Patent Application No. 10-2024-0029228, filed on Feb. 28, 2024 in the Korean Intellectual Property Office, and all the benefits accruing therefrom under 35 U.S.C. § 119, the disclosure of which in its entirety is incorporated herein by reference.

### BACKGROUND

### 1. Field

The disclosure relates to a surface analysis system for an air purification unit, and an air purification device including the surface analysis system for an air purification unit.

### 2. Description of the Related Art

To remove pollutants present in air methods of adsorption or removal of gas pollutants using adsorbents, for example, activated carbon having large specific surface area have been used, e.g., in air cleaning filters. However, with such adsorption or removal methods the previously adsorbed gas pollutants upon desorption may cause secondary pollution, e.g., exhausted adsorbents that undergo separate recycling processes such as heating adsorbents to high temperatures to release the adsorbed pollutants must be carefully controlled before the adsorbents can be reused. In some instances, the performance of adsorbents may sharply deteriorate in conditions where water or high humidity is present.

To address the above technical issues with using adsorption/removal methods, various catalysts may be used to remove pollutants from the air. The catalytic techniques, which use methods of decomposing/converting pollutants into harmless materials, may be used with minimal secondary pollution.

### SUMMARY

A surface analysis system for an air purification unit, the surface analysis system is capable of obtaining information regarding a surface of the air purification unit.

The surface analysis system for an air purification unit being capable of obtaining information of oxidation states (numbers) of a metallic catalyst using infrared spectroscopy.

Additional aspects will be set forth in part in the description which follows and, in part, will be apparent from the description, or may be learned by practice of the presented embodiments of the disclosure.

According to an aspect of the disclosure, a surface analysis system for an air purification unit includes an air purification unit including a carrier and a metallic catalyst supported on the carrier, wherein the air purification unit is configured to provide an air flow path in a first direction, a first light-source unit configured to irradiate inspection light onto a surface of the air purification unit, and an analysis unit configured to receive the inspection light reflected by the surface of the air purification unit and determine information regarding the surface of the air purification unit. The inspection light includes light with an infrared wavelength, and the analysis unit is further configured to obtain information on the oxidation number (states) of the metallic catalyst by infrared spectroscopy.

The analysis unit may be further configured to determine information of oxidation states (numbers) of the metallic catalyst located at a surface depth of at least about 1 nm but not more than about 9 nm from the surface of the air purification unit.

The surface analysis system may further include a second light-source unit configured to irradiate active light onto the surface of the air purification unit, particularly, if the metallic catalyst includes a photoactive catalyst.

A first light-source unit may be further configured to irradiate the inspection light in a second direction onto the surface of the air purification unit, the second direction intersecting with the first direction.

The surface analysis system may further include a partial pressure adjuster that is configured to provide a partial vacuum to the air flow path or supply an inert gas to the air flow path.

The surface analysis system may further include a temperature adjuster configured to heat or cool the air flow path.

The surface analysis system may further include a supply adjuster configured to supply polluted air, which is to be purified by the air purification unit, to the air flow path.

The surface analysis system may further include a control unit to control at least one of the partial pressure adjuster, the temperature adjuster, or the supply adjuster, depending on the information regarding the surface of the air purification unit, the information being determined by the analysis unit.

The control unit may be further configured to control at least one of the partial pressure adjuster, the temperature adjuster, and the supply adjuster by determining the oxidation states (numbers) of the metallic catalyst.

The metallic catalyst may include an oxide including copper.

The oxidation state number information, which is determined by the control unit, of the metallic catalyst may include the information of copper oxidation states and/or the copper metal. For example, the analysis unit may be configured to obtain information of elemental copper having an oxidation number of 0.

The oxidation number information of the copper atom having an oxidation number of 0 is determined by the control unit.

The first light-source unit may be further configured to continuously irradiate the inspection light onto the surface of the air purification unit as the air purification unit is operated to purify polluted air.

The analysis unit may be further configured to determine the information regarding the surface of the air purification unit in real time as the air purification unit is operated to purify polluted air.

The polluted air may include volatile organic compounds (VOCs) including at least one of gaseous formaldehyde, gaseous ammonia, gaseous acetaldehyde, gaseous acetic acid, and gaseous toluene.

The carrier may include a porous ceramic support.

The carrier may include a honeycomb structure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic diagram of a surface analysis system for an air purification unit, according to an embodiment;

FIG. 2 is a perspective schematic representation of an air purification unit according to an embodiment;

FIG. 3 is a block diagram illustrating operation of a first light-source unit and an analysis unit according to an embodiment;

FIG. 4A is an IR spectrum of a surface of an air purification unit according to an experimental example;

FIG. 4B is a software resolution IR spectrum obtained by normalizing or curve fitting the IR spectrum of FIG. 4A;

FIG. 4C is a software resolution IR spectrum obtained by normalizing or curve fitting the IR spectrum of FIG. 4A;

FIG. 5A is a photoelectron spectroscopy (XPS) plot of CuO and Cu₂O supported on TIO₂ as comparative examples;

FIG. 5B is a Raman spectrum of an air purification unit of the comparative examples of FIG. 5A;

FIG. 6A is a software resolution IR spectrum of an air purification unit, the IR spectrum being obtained through analysis using a partial pressure of 5 torr;

FIG. 6B is a software resolution IR spectrum of an air purification unit, the IR spectrum being obtained through analysis using a partial pressure of 10 torr;

FIG. 6C is a software resolution IR spectrum of an air purification unit, the IR spectrum being obtained through analysis using a partial pressure of 100 torr;

FIG. 7 is a block diagram illustrating a second light-source unit according to an embodiment;

FIG. 8 is a schematic illustration of an operational chemical processes with a second light-source unit;

FIG. 9A is a software resolution IR spectrum of an air purification unit before irradiation, the IR spectrum being obtained through analysis according to an experimental example;

FIG. 9B is a software resolution IR spectrum of an air purification unit following irradiation, the IR spectrum being obtained through analysis according to an experimental example;

FIG. 10A is a software resolution IR spectrum of an air purification unit before irradiation, the IR spectrum being obtained through analysis according to an experimental example;

FIG. 10B is software resolution IR spectra of an air purification unit over varying irradiation time, the IR spectra being obtained through analysis according to an experimental example; and

FIG. 11 is a diagram illustrating a pre-filter according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. In the drawings, the sizes of components may be exaggerated for clarity and convenience. The embodiments described below are only examples, and various modifications may be made to these embodiments. Accordingly, the embodiments are merely described below, by referring to the figures, to explain aspects of the present description.

As used herein, the term “and/or” includes any and all combinations of one or more of the associated listed items. Expressions such as “at least one of,” when preceding a list of elements, modify the entire list of elements and do not modify the individual elements of the list.

It will be understood that, when an element is referred to as being placed “on” another element, it can be placed directly on an upper, lower, left, or right surface of the other element, or intervening layer(s) may be present. In contrast, when an element is referred to as being “directly on” another element, there are no intervening elements present.

As used herein, the singular terms “a,” “an” and “the” are intended to include the plural forms as well, unless the context clearly indicates otherwise. Therefore, reference to “an” element in a claim followed by reference to “the” element is inclusive of one element as well as a plurality of the elements.

It will be understood that the terms “including” or “includes”, or “comprising” or “comprises” when used in this specification, specify the presence of stated features, regions, integers, steps, operations, elements, and/or components, and do not preclude other components, unless clearly stated otherwise.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

When a sequence of steps (or operations) of a method is clearly specified, or unless otherwise stated, the steps (or operations) may be performed in an appropriate sequence and are not limited to the described sequence. In addition, the term such as “ . . . unit (or portion)” or “ . . . module” used herein refers to a unit that processes at least one function or operation, and such a unit or module may be implemented by hardware, software, or a combination of hardware and software. Line-connections or connecting members between components, shown in the drawings, are illustrated as examples for functional connections and/or physical or circuit connections, and connections between components in an actual device may be represented by replaceable or additional various functional connections, physical connections, or circuit connections. All examples or terms used herein are only for describing the disclosure in detail, and the scope of the disclosure is not limited by these examples or terms unless defined by the accompanying claims.

The term “information on oxidation states of the metallic catalyst” refers to the relative presence of (or in some instance, relative amounts of) the different oxidation states of a particular catalytic metal including the metal in its elemental state at the surface as well as a depth distance from the surface of the catalyst.

Hereinafter, a surface analysis system for an air purification unit, and an air purification device, according to an embodiment, are described in detail.

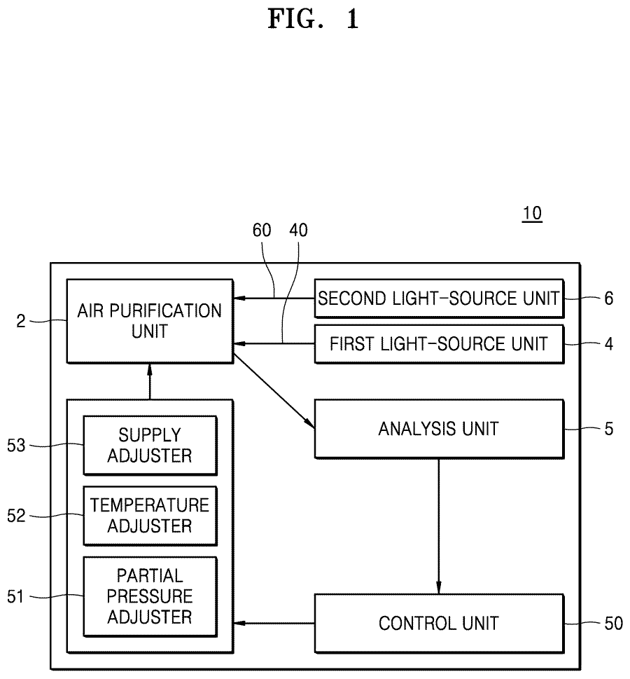

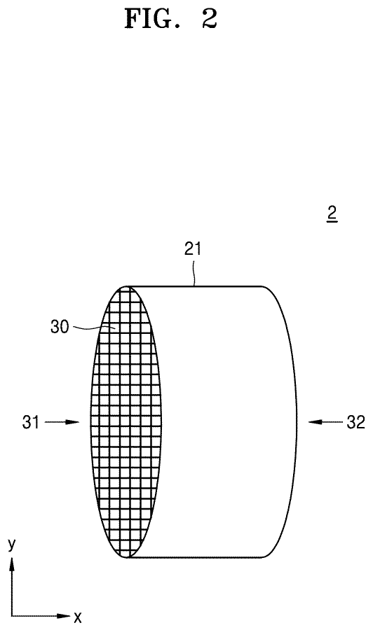

FIG. 1 is a block diagram of a surface analysis system 10 for an air purification unit 2, according to an embodiment. FIG. 2 is a perspective schematic representation of the air purification unit 2, according to an embodiment. Referring to FIGS. 1 and 2, the surface analysis system 10 for the air purification unit 2 may include the air purification unit 2 configured to purify polluted air. The polluted air may include at least one of a volatile organic compound (VOC) and/or carbon monoxide.

The VOC that is in the polluted air to be purified by the air purification unit 2 may include, for example, a polar compound, a nonpolar compound, or a combination thereof. The nonpolar compound may include, for example, an aliphatic hydrocarbon, an aromatic hydrocarbon, or a combination thereof. The aliphatic hydrocarbon and the aromatic hydrocarbon may each be substituted or unsubstituted with a substituent. The substituent may include, for example, an alkyl group, an alkenyl group, an alkynyl group, a cycloalkyl group, an aryl group, a heteroaryl group, a cycloalkenyl group, a heterocyclic group, a halogen, or a combination thereof. The aliphatic hydrocarbon may include, for example, methane, ethane, propane, butane, pentane, hexane, or a combination thereof. The aromatic hydrocarbon may include, for example, benzene, toluene, xylene, or a combination thereof.

The VOC may include, for example, a polar compound. The polar compound may include, for example, ammonia (NH₃), an amine compound, an aldehyde compound, a ketone compound, an alcohol compound, a sulfur compound, a thiol compound, a halogenated hydrocarbon, nitrogen oxide (NOₓ), sulfur oxide (SOₓ), ozone, or a combination thereof. The amine compound may include, for example, methylamine, dimethylamine, trimethylamine, ethylamine, aniline, or a combination thereof. The aldehyde compound may include, for example, formaldehyde, acetaldehyde, propiolaldehyde, butyraldehyde, or a combination thereof. The ketone compound may include, for example, dimethyl ketone, methyl ethyl ketone, diethyl ketone, methyl propyl ketone, dipropyl ketone, or a combination thereof. The alcohol compound may include, for example, methanol, ethanol, propanol, isopropanol, butanol, pentanol, hexanol, heptanol, or a combination thereof. The sulfur compound may include, for example, hydrogen sulfide, sulfur dioxide, elemental sulfur, a sulfur oxide (SOₓ), or a combination thereof. The thiol compound may include, for example, methanethiol, ethanetyrol, 1-propanethiol, 2-propanethiol, propenethiol, butanethiol, tert-butyl mercaptan, thiopetol, or a combination thereof.

A method performed by the air purification unit 2 may include a catalyst. The catalyst may refer to various catalysts, such as a photocatalyst, a surface catalyst, an organic catalyst, and the like. In addition, purifying polluted air by the air purification unit 2 may refer to converting one or more VOCs into gases that are, in general, harmless to humans. However, the method performed by the air purification unit 2 for purifying polluted air is not limited to a purification method in the presence of a catalyst. For example, the method, performed by the air purification unit 2 may include a method of purifying polluted air by applying heat, pressure, or the like to the polluted air.

The air purification unit 2 may correspond to a component provided to an air purification device. In other words, the air purification unit 2 may be arranged in the air purification device such that the air purification unit 2 may purify polluted air. However, the disclosure is not limited to the example described above, and the air purification unit 2 may independently purify polluted air regardless of the air purification device.

The air purification unit 2 according to an embodiment may be configured as a module of an air purification device. More specifically, the air purification unit 2 may be provided in a form such that the air purification unit 2 may be a replaceable module in the air purification device. However, the disclosure is not limited thereto, and the air purification unit 2 may be configured as a semi-permanent unit that is without the need for replacement over an extended period of time.

Referring to FIG. 2, the air purification unit 2 according to an embodiment may include a carrier 21. An element constituting the carrier 21 may include one or more of Al, Si, Zr, and Ti. The carrier 21 may include a metal oxide. For example, the carrier 21 may include at least one of AlₓO_{y} (0<x≤2, 0<y≤3), SiO_{y} (0<y≤2), ZrO_{y} (0<y≤2), and TiO_{y} (0<y≤2). More specifically, the carrier 21 may include at least one of Al₂O₃, SiO₂, ZrO₂, and TiO₂. In particular, the carrier 21 may include, but is not limited to, TiO₂. For example, the carrier 21 may include a ceramic material.

The carrier 21 according to an embodiment may be configured for a metallic catalyst described below to be easily supported on the carrier 21. For example, the carrier 21 may include, but is not limited to, a porous ceramic support. The carrier 21 may be configured to have a large area capable of contacting polluted air. The carrier 21 may be configured for polluted air to efficiently flow in an air flow path 30. For example, the carrier 21 may include, but is not limited to, a honeycomb structure, or another high surface are structure.

The air purification unit 2 according to an embodiment may include an entry region 31 or front end through which polluted air flows into the air purification unit 2, and a discharge region 32 or back end through which purified air is discharged from the air purification unit 2. Polluted air may flow toward the entry portion 31 and may be purified inside the air purification unit 2. The air purification unit 2 may purify polluted air and may discharge purified air toward the discharge portion 32. The entry portion 31 and the discharge portion 32 may be respectively arranged side portions of the air purification unit 2 in a first direction. Here, the first direction may be a direction parallel to an x-axis direction as indicated. However, the aforementioned arrangements of the entry portion 31 and the discharge portion 32 are only examples, and the disclosure is not limited thereto.

The air purification unit 2 according to an embodiment may include the air flow path 30, the air flow path 30 proceeds in the first direction, i.e., from the entry portion 31 and the discharge portion 32. The formation of the air flow path 30 in the first direction may mean that a certain cross-sectional shape of the air flow path 30 formed for polluted air to flow through the air purification unit 2 extends in the first direction. The air flow path 30, which is formed in the first direction in the air purification unit 2, and may be substantially flow in the first direction in the carrier 21. However, a plurality of air flow paths 30 may be provided. Polluted air may flow in the first direction through the air flow path 30 that is formed in the first direction in the carrier 21. However, the above description of the air flow path 30 is provided only as an example, and the disclosure is not limited thereto. For example, the air purification unit 2 may include an air flow path 30 extending in a curved direction.

The air purification unit 2 according to an embodiment may include a metallic catalyst that is supported on the carrier 21. The metallic catalyst may be chemically bonded to the carrier 21, but the disclosure is not limited thereto. The metallic catalyst being supported on the carrier 21 may mean that the carrier 21 supports the metallic catalyst.

The metallic catalyst may comprise one or more transition metals. The metallic catalyst may comprise an oxide of a transition metal. For example, the metallic catalyst may comprise an oxide of copper. More specifically, the metallic catalyst may comprise at least one of Cu⁰, Cu₂O, or CuO. Moreover, the metallic catalyst may comprise a metal element in a non-oxidized (elemental) form in addition to the metal as a metal oxide. In other words, the metallic catalyst may include a metal element having an oxidation number of 0, e.g., Cu⁰.

In accordance with an embodiment, the metallic catalyst may include elemental copper with an oxidation number of 0. The metal element constituting the metallic catalyst may have various oxidation numbers. The metallic catalyst may include the same metal with different oxidation numbers (states). For example, the metallic catalyst may include copper having at least two of the oxidation numbers of +2, +1, and 0. However, the above description of the oxidation number of the metal in the metallic catalyst is provided only as an example, and the disclosure is not limited thereto.

The oxidation number of the metal in the metallic catalyst according to an embodiment may vary as the air purification unit 2 purifies polluted air. More specifically, a relative proportion of a metal having a different oxidation number in the metallic catalyst may vary during operation of the surface analysis system 10 as the air purification unit 2 purifies polluted air. For example, if the metallic catalyst comprises copper in at least two different oxidation numbers of +2, +1, and 0, the respective proportions of copper in the different oxidation numbers of +2, +1, and 0 may vary as the air purification unit 2 purifies polluted air.

In the metallic catalyst the respective proportions of a metal in its different oxidation numbers may influence the performance of the air purification unit 2. For example, if the metallic catalyst includes copper in at least two oxidation numbers of +2, +1, and 0, as the proportion of the copper in the catalyst having a low oxidation number increases, the performance of the air purification unit 2 to purify polluted air may increase. However, the above description of the relationship between the performance of the air purification unit 2 and the oxidation number of the metal element in the metallic catalyst is provided only as an example, and the disclosure is not limited thereto.

Polluted air flowing in the air purification unit 2 may contact a surface of the air purification unit 2. The surface of the air purification unit 2 may correspond to a surface of the metallic catalyst supported on the carrier 21. Polluted air may contact the surface of the metallic catalyst. The oxidation number of the metal element located at the surface of the metallic catalyst may influence the performance of the air purification unit 2 to purify polluted air. The oxidation number of the metal element proximate to the surface of the metallic catalyst may be appropriately adjusted or maintained, and thereby, the performance or efficiency of the air purification unit 2 to purify polluted air may improve.

Hereinafter, the surface analysis system 10 for the air purification unit 2 is described, the surface analysis system 10 being capable of determining the oxidation number of the metal element, e.g., copper, proximate to the surface of the metallic catalyst.

Referring to FIGS. 1, 2, and 3, the surface analysis system 10 for the air purification unit 2, may include a light-source unit 4. The first light-source unit 4 may be configured to irradiate inspection light 40 onto the surface of the air purification unit 2. The inspection light 40 irradiated by the first light-source unit 4 may include an electromagnetic wave for inspecting the surface of the air purification unit 2. The inspection light 40 may include, but is not limited to, an electromagnetic wave having an infrared wavelength. Here, the surface of the air purification unit 2 may correspond to, but is not limited to, the surface of the air purification unit 2 inside the air flow path 30. In addition, the surface of the air purification unit 2 may correspond to, but is not limited to, the surface of the metallic catalyst.

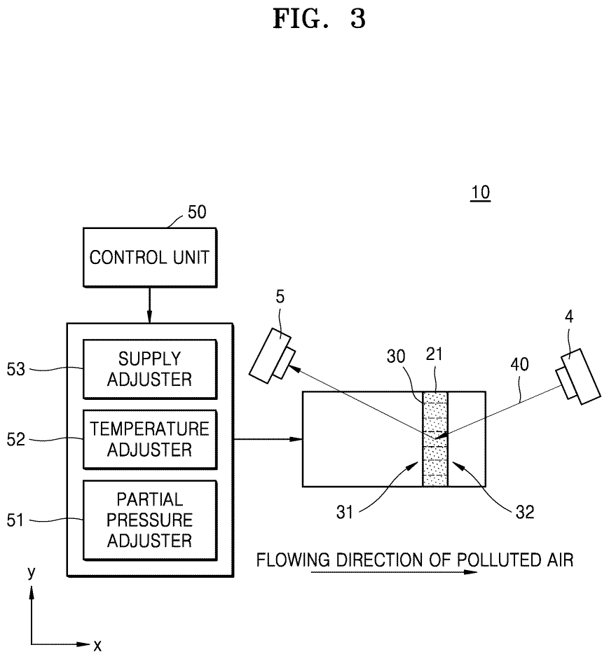

FIG. 3 is a diagram illustrating operations of a first light-source unit 4 and an analysis unit 5, according to an embodiment. The inspection light 40 irradiated by the first light-source unit 4 may be reflected by the surface of the air purification unit 2. The reflection of the inspection light 40 by the surface of the air purification unit 2 may refer to a portion of the inspection light 40 that is not absorbed by the surface of the air purification unit 2 and is reflected by the surface of the air purification unit 2. The first light-source unit 4 may irradiate the inspection light 40 in a second direction intersecting with the first direction of the air flow path 30. For example, the irradiation of the inspection light 40 may be directed in a direction intersecting, e.g., near perpendicular (or about 70° to 90°) with the direction of the air flow path 30 for analysis of the reflected inspection light. However, the direction in which the first light-source unit 4 irradiates the inspection light 40, and a moving path of the inspection light 40 are not limited to the example described above.

The surface analysis system 10 for the air purification unit 2, according to an embodiment, may include the analysis unit 5. The analysis unit 5 may be configured to receive a portion of the inspection light 40 that is reflected by the surface of the air purification unit 2. The analysis unit 5 may obtain information regarding the surface of the air purification unit 2 by analyzing the inspection light 40. The analysis unit 5 may determine information regarding the surface of the air purification unit 2 by analyzing the inspection light 40 using infrared spectroscopy (that is, Fourier-transform infrared spectroscopy (FT-IR)). The information regarding the surface of the air purification unit 2 may include information of the variation in oxidation numbers of the metallic catalyst. For example, the information regarding the surface of the air purification unit 2 may include the oxidation number information of the metallic catalyst located at a surface depth of 9 nm or less, or 5 nm or less, from the surface of the air purification unit 2. The information regarding the surface of the air purification unit 2 may include information of the oxidation number information of the metallic catalyst located at a surface depth of at least about 1 nm but not more than about 9 nm, or about 1 nm but not more than about 5 nm, from the surface of the air purification unit 2. The aforementioned ranges of surface depths of the air purification unit 2 are provided only as examples, and the disclosure is not limited thereto. The location of the metallic catalyst, from which the analysis unit 5 may obtain the variation in oxidation number of the metallic catalyst. which substantially has a possibility of chemically influencing the efficiency of the air purification unit 2 to purify air. The oxidation number information may be included in a range of information determined by the analysis unit 5.

The surface analysis system 10 for the air purification unit 2, may evaluate the polluted-air purification performance of the air purification unit 2 from the analysis unit 5, e.g., the variation in oxidation number information of the metallic catalyst. In other words, the analysis unit 5 may evaluate the polluted-air purification performance of the air purification unit 2 by analyzing an oxidation number variation (or distribution) of the metal element in the metallic catalyst. Here, the oxidation number distribution of the metal element refer to the metal having a plurality of oxidation numbers, the respective proportions of the metal in each oxidation state including the elemental metal.

In the surface analysis system 10 for the air purification unit 2, the first light-source unit 4 may continuously irradiate the surface of the air purification unit 2 with the inspection light 40, as the air purification unit 2 is operated to purify polluted air. In other words, a process of purifying polluted air by the air purification unit 2, and a process of irradiating with the first light-source unit 4 the inspection light 40 onto the surface of the air purification unit 2 may be simultaneously (or continuously) performed.

The analysis unit 5 according to an embodiment may continuously determine the information regarding the surface of the air purification unit 2 by receiving the inspection light 40 reflected by the surface of the air purification unit 2, as the air purification unit 2 is operated to purify polluted air. In other words, the analysis unit 5 may determine the information regarding the surface of the air purification unit 2 in real time. Accordingly, the analysis unit 5 may analyze the polluted-air purification performance of the air purification unit 2 in the process in which the air purification unit 2 purifies polluted air.

The surface analysis system 10 for the air purification unit 2, may include a partial pressure adjuster 51 configured to selectively form a partial vacuum and/or supply an inert gas to the air purification unit 2, e.g., to maintain a select partial pressure in the air purification unit 2. The partial pressure adjuster 51 may selectively provide a vacuum or supply an inert gas to the air flow path 30 arranged in the air purification unit 2. The partial pressure adjuster 51 may control a pressure condition in the air purification unit 2 during operation.

The surface analysis system 10 for the air purification unit 2, may include a temperature adjuster 52 configured to heat or cool the air purification unit 2. For example, the temperature adjuster 52 may include heating or cooling of the air purification unit 2 by adjusting the temperature of the air flow path 30. The temperature adjuster 52 may control a temperature condition in which the air purification unit 2 purifies polluted air. However, the function of the temperature adjuster 52 is not limited to the example described above.

The surface analysis system 10 may include a supply adjuster 53 configured to supply or control the amount (volume) of polluted air that is directed to the air purification unit 2. The air purification unit 2 may be supplied with polluted air by the supply adjuster 53. The supply adjuster 53 may adjust the amount of the polluted air supplied to the air purification unit 2. The supply adjuster 53 may adjust components of the polluted air supplied to the air purification unit 2. The supply adjuster 53 may adjust the polluted-air purification efficiency of the air purification unit 2 by adjusting the supply of the polluted air. However, the function of the supply adjuster 53 is not limited to the example described above.

The surface analysis system 10 may include a control unit 50 configured to selectively control at least one of the partial pressure adjuster 51, the temperature adjuster 52, or the supply adjuster 53. The control unit 50 may monitor and adjust at least one of the partial pressure adjuster 51, the temperature adjuster 52, and the supply adjuster 53, and thereby, improve the efficiency of the air purification unit 2 to purify polluted air.

The analysis unit 5 may transfer the information regarding the surface of the air purification unit 2 to the control unit 50. The control unit 50 may receive the information regarding the surface of the air purification unit 2, and/or the information being obtained by the analysis unit 5.

The control unit 50 may selectively control at least one of the partial pressure adjuster 51, the temperature adjuster 52, or the supply adjuster 53, depending on the information regarding the surface of the air purification unit 2. For example, the control unit 50 may selectively control at least one of the partial pressure adjuster 51, the temperature adjuster 52, and the supply adjuster 53, depending on the oxidation number information of the metallic catalyst received from the analysis unit 5. However, the function of the control unit 50 is not limited to the example described above.

Hereinafter, experimental examples and comparative examples, in which analysis experiments were performed by using the surface analysis system 10 for the air purification unit 2, according to an embodiment, are described in detail.

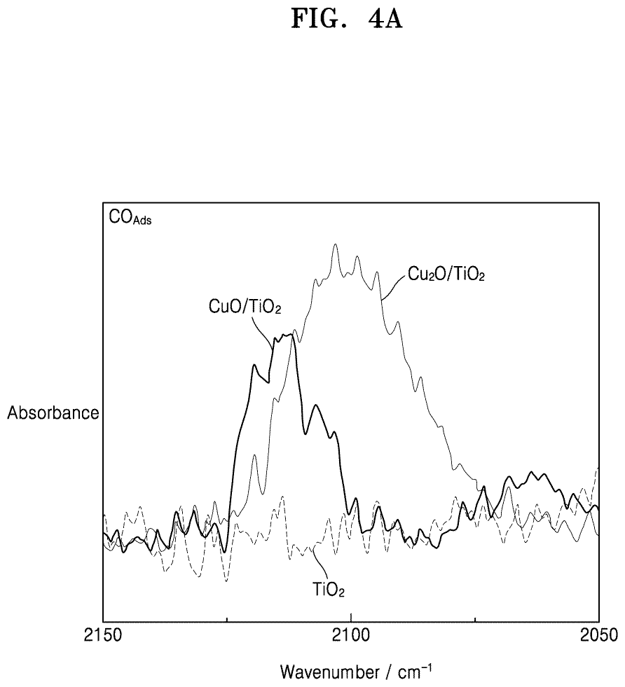

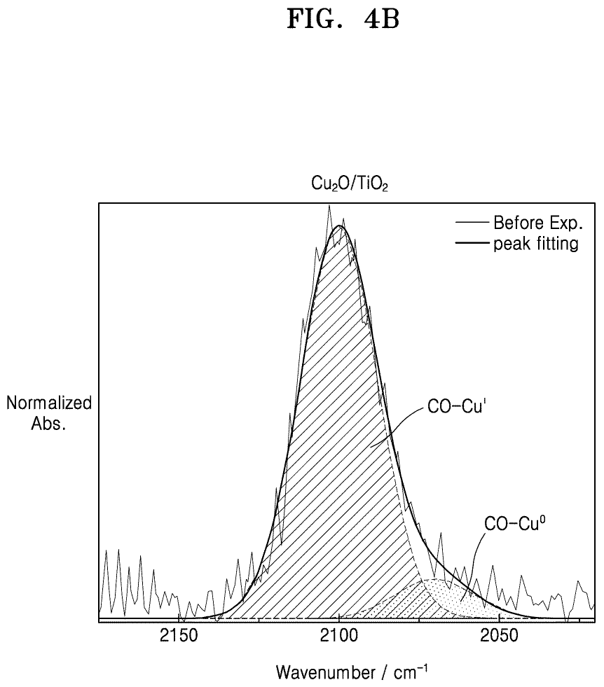

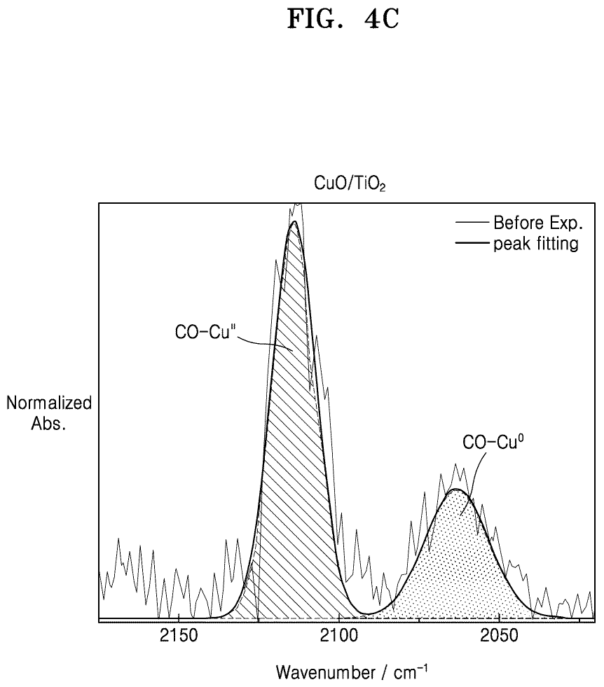

FIG. 4A is a graph of a component distribution of the air purification unit 2, the graph obtained through the surface analysis of the unit 2 according to an experimental example. FIG. 4B is a graph obtained by normalizing the graph of FIG. 4A. FIG. 4C is a graph obtained by normalizing the graph of FIG. 4A. The graphs of FIGS. 4A to 4C are obtained from an experimental example in which analysis experiments are performed by using the surface analysis system 10 for the air purification unit 2, according to an embodiment.

More specifically, in the experimental example of FIGS. 4A to 4C, the air purification unit 2 is configured to adsorb carbon monoxide, and the information regarding the surface of the air purification unit 2 is obtained through infrared spectroscopy. In the graph of FIG. 4A, the horizontal axis represents wavenumbers, and the vertical axis represents relative indicators indicating the degree of adsorption of carbon monoxide. The carrier 21 of the air purification unit 2, which is used in the experimental example, may include TiO₂. Experiments were performed under different conditions of the metallic catalyst, and the conditions of the metallic catalyst are divided into a condition including Cu₂O and a condition including CuO. FIG. 4A illustrates the respective degrees of adsorption of carbon monoxide by the metallic catalyst including Cu₂O, the metallic catalyst including CuO, and TiO₂, overlapped on the same graph for comparison. The horizontal axis of the graph of FIG. 4A represents wavenumbers in units of cm⁻¹. FIG. 4B is a graph obtained by normalizing the experimental graph for the metallic catalyst including Cu₂O. FIG. 4C is a graph obtained by normalizing the experimental graph for the metallic catalyst including CuO.

Referring to FIGS. 3 to 4C, it may be confirmed that the surface analysis system 10 for the air purification unit 2, according to the experimental example, may obtain the information regarding the surface of the air purification unit 2. More specifically, the information regarding the surface of the air purification unit 2 may be obtained by analyzing the adsorption distribution of carbon monoxide adsorbed on the surface of the air purification unit 2. In particular, it may be confirmed that both the metallic catalyst including Cu₂O and the metallic catalyst including CuO detect copper having an oxidation number of 0.

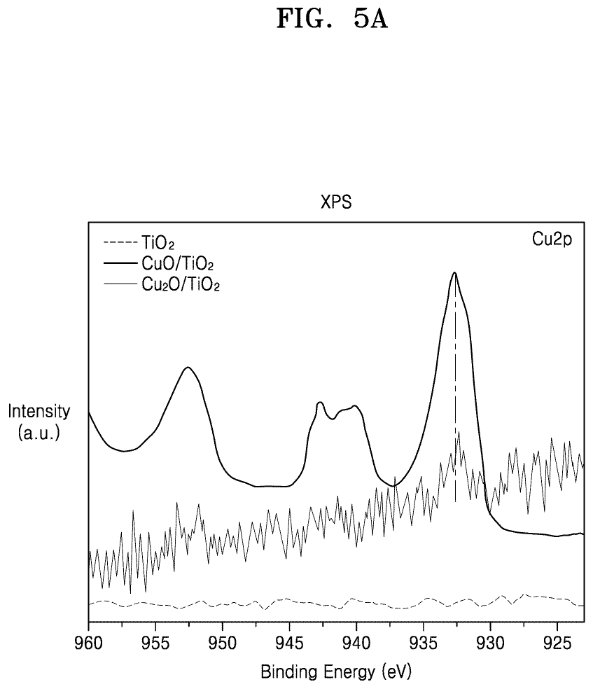

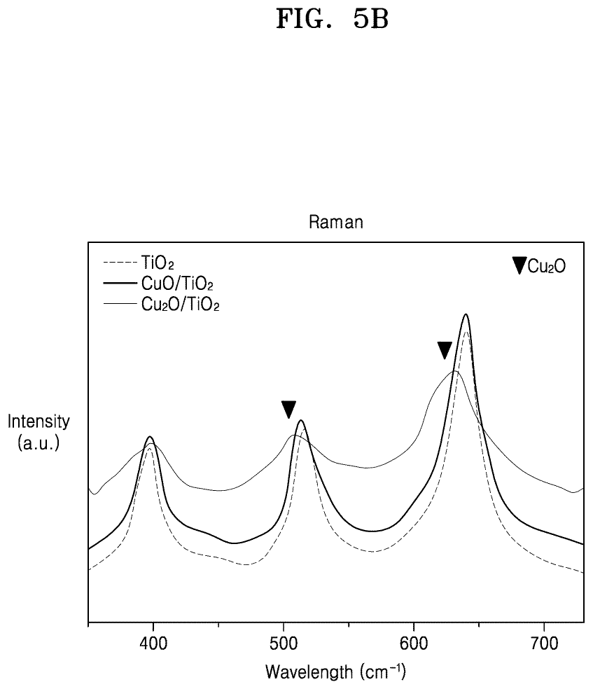

FIG. 5A is a graph of a component distribution of the air purification unit 2, the graph being obtained through X-ray photoelectron spectroscopy (XPS), as a comparative example. FIG. 5B is a graph of a component distribution of the air purification unit 2, the graph being obtained through Raman spectroscopy, as a comparative example. Experiments were performed under different conditions of the metallic catalyst, and the conditions of the metallic catalyst are divided into conditions including Cu₂O and conditions including CuO. FIGS. 5A and 5B each illustrate the respective degrees of adsorption of carbon monoxide by the metallic catalyst including Cu₂O, the metallic catalyst including CuO, and TiO₂, on the same graph for comparison.

Referring to FIGS. 3 to 5B, it may be confirmed that an analysis system using XPS and Raman spectroscopy is unable to detect copper having an oxidation number of 0. In addition, it may be confirmed that the analysis system using XPS and Raman spectroscopy is unable to continuously track changes in the surface of the air purification unit 2. On the other hand, the surface analysis system 10 for the air purification unit 2, according to the experimental example, may detect copper having an oxidation number of 0 as well as continuously track changes in the surface of the air purification unit 2, because unlike the comparative example using XPS and Raman spectroscopy, the surface analysis system 10 for the air purification unit 2 uses infrared spectroscopy to obtain information regarding relatively shallow surfaces, that is, extreme surfaces. In other words, in the experimental example of the surface analysis system 10 for the air purification unit 2, which obtains the information regarding the surface of the air purification unit 2 through infrared spectroscopy, the oxidation number of the metallic catalyst located on an extreme surface of the air purification unit 2 may be obtained, thereby detecting copper having an oxidation number of 0.

The surface analysis system 10 for the air purification unit 2 may analyze the surface of the air purification unit 2 as the control unit 50, my monitor and adjust, for example, the partial pressure adjuster 51.

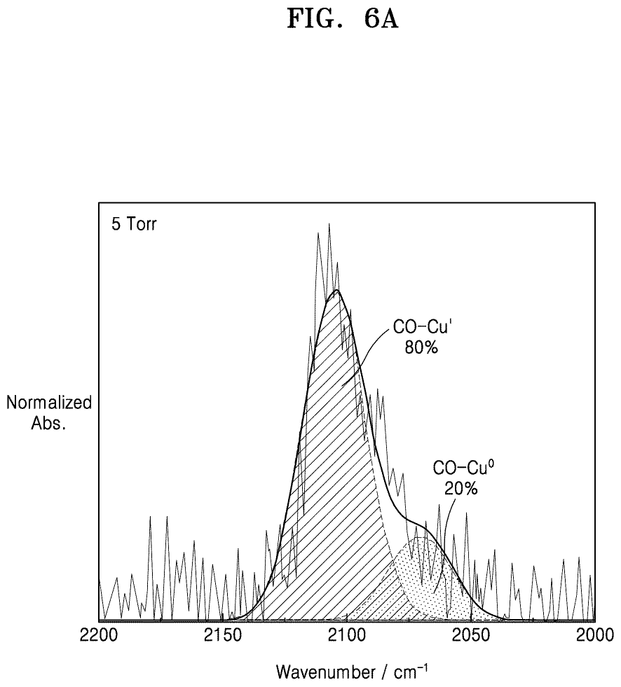

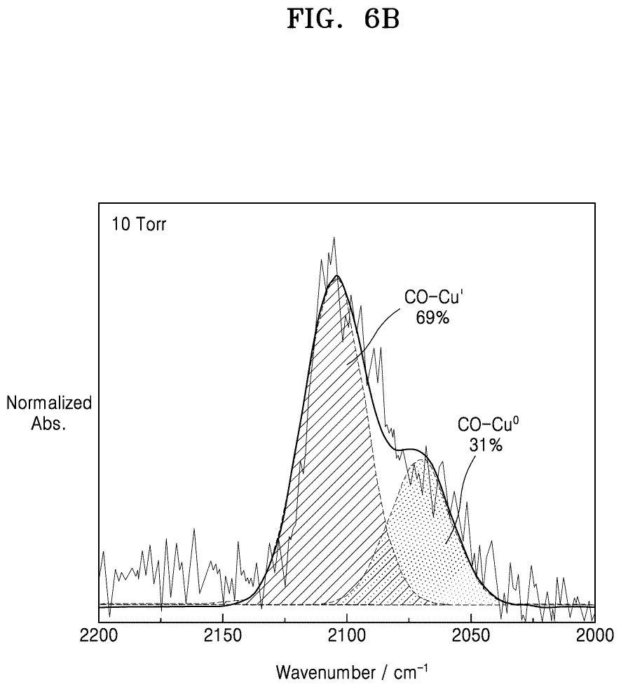

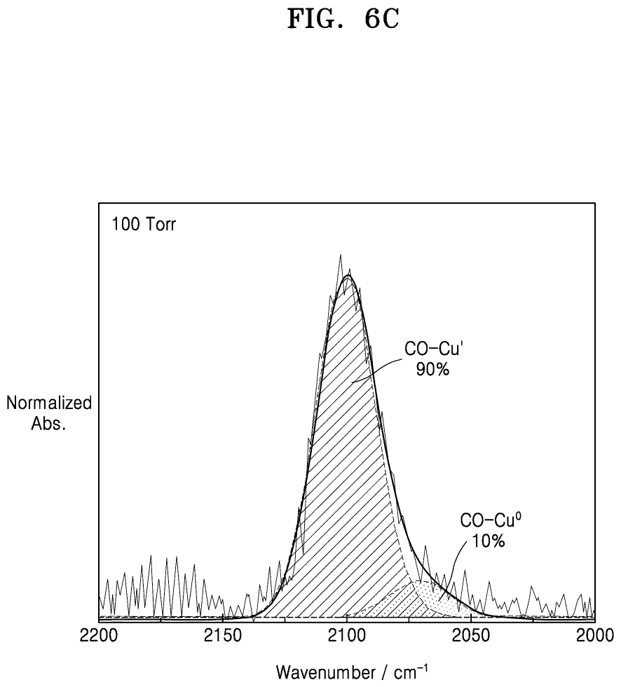

FIG. 6A is an IR spectrum of a surface of the air purification unit 2, the spectrum being obtained through analysis using a different partial pressure, according to an experimental example. FIG. 6B is IR spectrum of a surface of the air purification unit 2, the IR spectrum being obtained through analysis using a different partial pressure, according to an experimental example. FIG. 6C is IR spectrum of a surface of the air purification unit 2, the IR spectrum being obtained through analysis using a different partial pressure, according to an experimental example. The carrier 21, which is used in the experimental examples, of the air purification unit 2 includes TiO₂. The metallic catalyst supported on the carrier 21 includes Cu₂O. Experiments were performed under different conditions of the partial pressure of carbon monoxide adsorbed on the surface of the air purification unit 2, and experimental results according to the partial pressures of 5 Torr, 10 Torr, and 15 Torr are shown in FIGS. 6A, 6B, and 6C, respectively.

Referring to FIGS. 3 to 6C, it may be confirmed that the oxidation number information varies in the experimental examples respectively performed under the different conditions of the partial pressure. More specifically, it may be confirmed that the proportion of the copper atom having an oxidation number of 0 in the metallic catalyst varies along with the varying conditions of the partial pressure of carbon monoxide.

The control unit 50 according to an embodiment may selectively control at least one of the partial pressure adjuster 51, the temperature adjuster 52, and the supply adjuster 53 by determining the oxidation number information of the metallic catalyst.

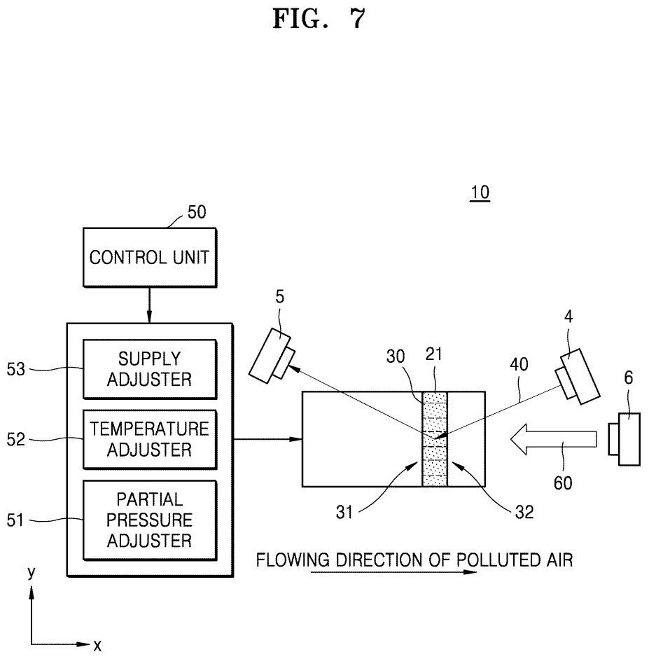

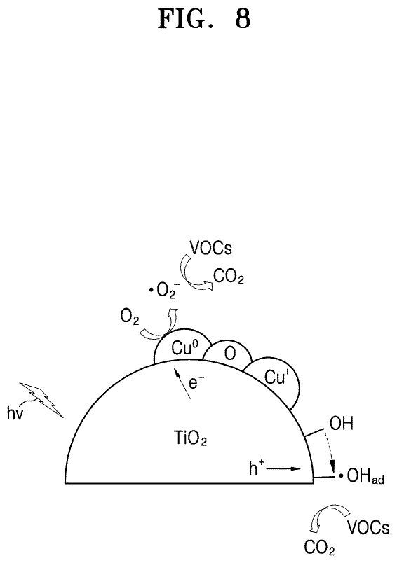

FIG. 7 is a diagram illustrating a second light-source unit 6 according to an embodiment. FIG. 8 is a diagram illustrating an operational principle of the second light-source unit 6. Referring to FIGS. 7 and 8, the surface analysis system 10 for the air purification unit 2, may include the second light-source unit 6. The second light-source unit 6 may be configured to irradiate active light 60 onto the surface of the air purification unit 2. The active light 60 onto the surface of the air purification unit 2 may include irradiating the active light 60 onto the air flow path 30 in the air purification unit 2. The second light-source unit 6 may include irradiating the active light 60 onto the metallic catalyst of the air purification unit 2. Here, the active light 60 may include, but is not limited to, an electromagnetic wave having an ultraviolet wavelength.

The metallic catalyst according to an embodiment may include a photoactive catalyst. The carrier 21 may include a photoactive catalyst. The metallic catalyst may be activated by the active light 60 irradiated by the second light-source unit 6. The carrier 21 may be activated by the active light 60 irradiated by the second light-source unit 6. The term “activated” means that the capability of purifying polluted air is improved. More specifically, activated means that, as energy equal to or greater than band gap energy is irradiated onto a surface and electrons transit from a valence band to a conduction band, pairs of electrons (e−) and holes (h+) are generated. Holes generated in the conduction band may contribute to an oxidation reaction and may generate hydroxyl radicals (OH) through reactions with water molecules adsorbed on the surface or may oxidize organic materials, for example, VOCs, through direct reactions with the organic materials. Electrons generated in the conduction band may form superoxide ions (O₂⁻) by causing reduction reactions of oxygen molecules and may generate hydroxyl radicals through several-stage additional reactions. The VOCs may be decomposed into carbon dioxide and water due to the hydroxyl radicals generated by the holes and the electrons.

The surface analysis system 10 for the air purification unit 2 may obtain the information regarding the surface of the air purification unit 2 in real time, as the second light-source unit 6 is irradiating the active light 60 onto the surface of the air purification unit 2, thereby obtaining information regarding the surface of the air purification unit 2 in real time. As a result, changes over time regarding the surface information of the air purification unit 2 may be continuously obtained and acted upon accordingly.

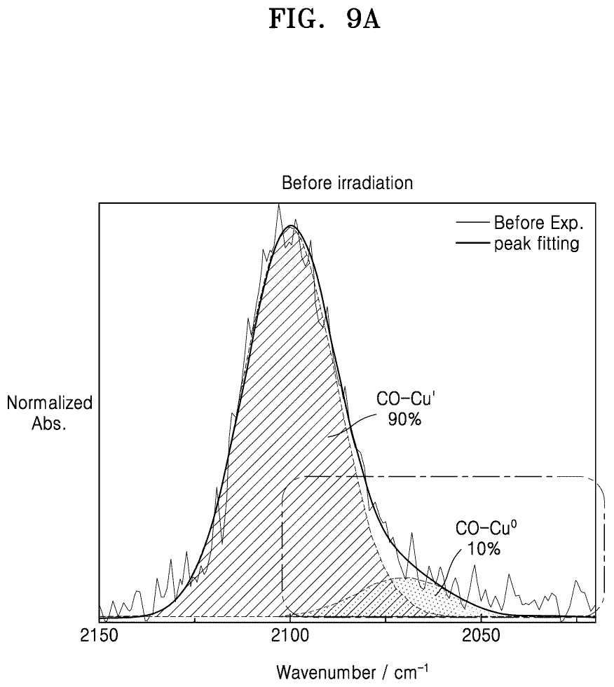

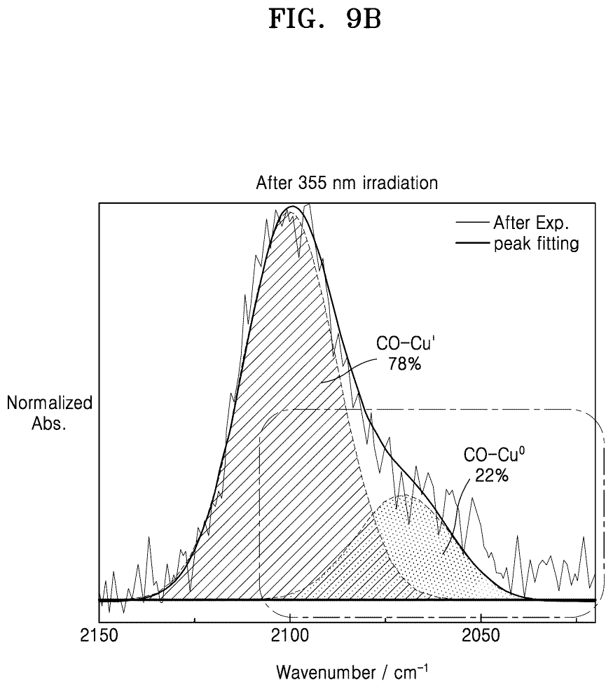

FIG. 9A is a software resolution IR spectrum of a surface of the air purification unit 2 prior to irradiation of active light 60 from the second light-source unit 6, the IR spectrum being obtained through analysis according to an experimental example. FIG. 9B is a software resolution IR spectrum of a surface of the air purification unit 2 following irradiation of active light 60, the IR spectrum being obtained through analysis according to an experimental example. More specifically, FIGS. 9A and 9B each illustrate the information regarding the surface of the air purification unit 2 by obtaining the information regarding the surface of the air purification unit 2 before and after the second light-source unit 6 irradiates the active light 60 onto the surface of the air purification unit 2. As indicated, the presence of CO adsorbed onto Cu⁰ increases following irradiation of active light 60 with a consequent loss of CO adsorbed onto Cu¹ (Cu₂O).

Referring to FIGS. 8 to 9B, it may be confirmed that, after the second light-source unit 6 irradiates the active light 60 onto the surface of the air purification unit 2, the proportion of zero-valent copper in the metallic catalyst is increased. The surface analysis system 10 for the air purification unit 2 may detect surface changes before and after the air purification unit 2 is activated, by analyzing the surface of the air purification unit 2 in real time.

The surface analysis system 10 for the air purification unit 2, according to an embodiment, may control the respective proportions of the oxidation numbers of the metal elements in the metallic catalyst by causing the second light-source unit 6 to irradiate the active light 60 onto the surface of the air purification unit 2. To control the respective proportions of the oxidation numbers of the metal elements, the surface analysis system 10 for the air purification unit 2 may control operations of the second light-source unit 6.

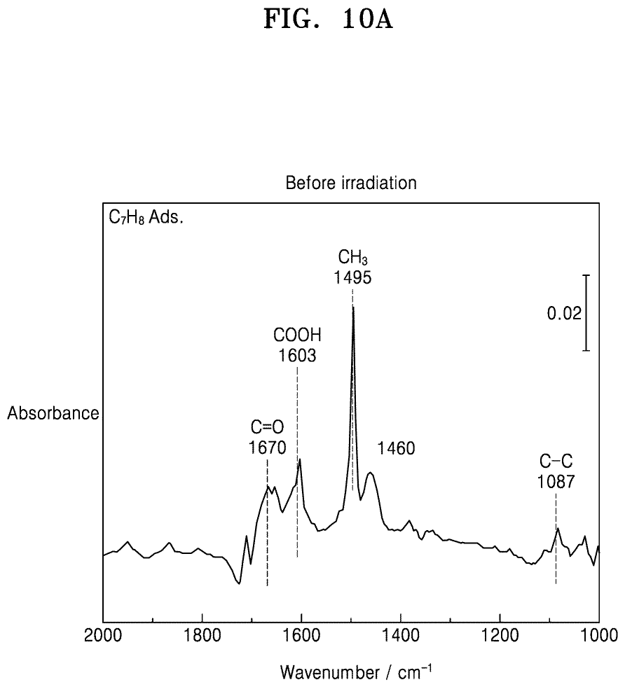

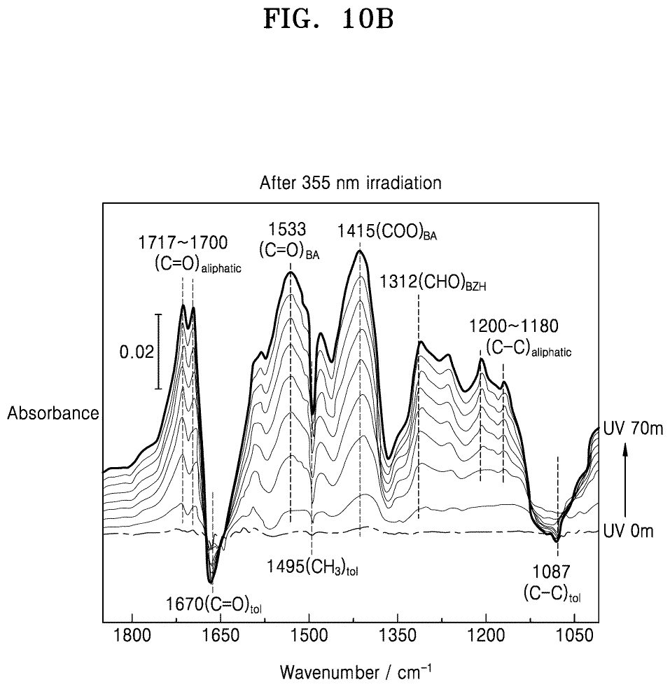

FIG. 10A is an IR spectrum of a surface of an air purification unit prior to irradiation of active light 60 from the second light-source unit 6, the IR spectrum obtained through analysis according to an experimental example. FIG. 10B is a overlap plot of IR spectra of a surface of an air purification unit following irradiation of active light 60 over time, the plot being obtained through analysis according to an experimental example. Each of the experimental examples of FIGS. 10A and 10B provides an IR spectra obtained by analyzing the surface of the air purification unit 2 in real time in the process in which the air purification unit 2 operates to purify air including toluene (C₇H₈). More specifically, FIGS. 10A and 10B each illustrate the information regarding the surface of the air purification unit 2 by obtaining the information regarding the surface of the air purification unit 2 before and after the second light-source unit 6 irradiates the active light 60 onto the surface of the air purification unit 2.

Referring to FIGS. 8, 10A, and 10B, it may be confirmed that following the second light-source unit 6 irradiation of active light 60 onto the surface of the air purification unit 2 the ability of the metallic catalyst to decomposetoluene increases. In particular, referring to FIG. 10B, the surface analysis system 10 for the air purification unit 2 may check the efficiency or rate of decomposition of toluene in real time by obtaining the information regarding the surface of the air purification unit 2 in real time.

The air purification unit 2 according to an embodiment may check the purification process of polluted air in real time. The air purification unit 2 may selectively control the control unit 50 and the second light-source unit 6 by determining the information regarding the surface of the air purification unit 2 in the purification process of polluted air in real time, thereby improving the purification efficiency of the air purification unit 2.

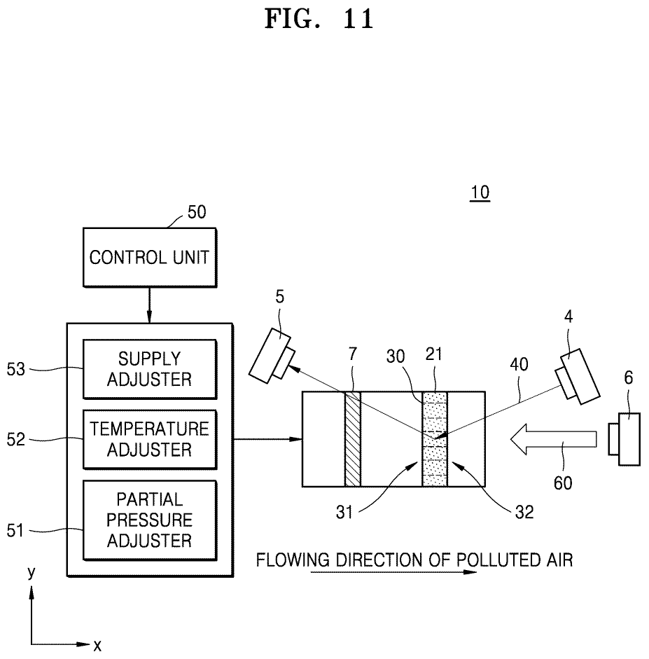

FIG. 11 is a diagram illustrating a pre-filter 7 according to an embodiment.

Referring to FIG. 11, the surface analysis system 10 for the air purification unit 2, according to an embodiment, may include a pre-filter 7 arranged on one side of the air purification unit 2. The pre-filter 7 may be arranged on an upstream side of the air purification unit 2 based on the flow direction of polluted air. The pre-filter 7 may be arranged downstream, i.e., opposite to the entry portion 31 for the polluted air. The pre-filter 7 may physically remove fine particles in the polluted air. The pre-filter 7 may include a filtering net capable of filtering out fine particles in the polluted air. However, the function and arrangement of the pre-filter 7 are not limited to the examples described above.

A surface analysis system for an air purification unit, according to an embodiment, may obtain oxidation number information of a metallic catalyst.

An air purification device according to an embodiment may have high efficiency in purifying polluted air.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A surface analysis system for an air purification unit, the surface analysis system comprising:
an air purification unit comprising a carrier and a metallic catalyst supported on the carrier, wherein the air purification unit is configured to provide an air flow path in a first direction;
a first light-source unit configured to irradiate inspection light onto a surface of the air purification unit; and
an analysis unit configured to receive the inspection light reflected by the surface of the air purification unit and determine information regarding the surface of the air purification unit,
wherein the inspection light includes light with an infrared wavelength, and the analysis unit determines information on oxidation number of the metallic catalyst by infrared spectroscopy.

2. The surface analysis system of claim 1, wherein the analysis unit is configured to obtain information on oxidation number information of the metallic catalyst proximate to a surface depth of at least about 1 nm but not more than about 9 nm from the surface of the air purification unit.

3. The surface analysis system of claim 2, further comprising a second light-source unit configured to irradiate active light onto the surface of the air purification unit,
wherein the metallic catalyst comprises a photoactive catalyst.

4. The surface analysis system of claim 3, wherein the first light-source unit is further configured to irradiate the inspection light in a second direction onto the surface of the air purification unit, the second direction intersecting with the first direction.

5. The surface analysis system of claim 3, further comprising a partial pressure adjuster to selectively form a vacuum in or supply an inert gas to the air flow path.

6. The surface analysis system of claim 5, further comprising a temperature adjuster to heat or cool the air flow path.

7. The surface analysis system of claim 6, further comprising a supply adjuster to supply polluted air, which is to be purified by the air purification unit, into the air flow path.

8. The surface analysis system of claim 7, further comprising a control unit to control at least one of the partial pressure adjuster, the temperature adjuster, and the supply adjuster, depending on the information regarding the surface of the air purification unit, the information being determined by the analysis unit.

9. The surface analysis system of claim 8, wherein the control unit controls at least one of the partial pressure adjuster, the temperature adjuster, and the supply adjuster by determining the oxidation number information of the metallic catalyst.

10. The surface analysis system of claim 9, wherein the metallic catalyst comprises an oxide comprising copper.

11. The surface analysis system of claim 10, wherein the oxidation number information, which is determined by the control unit, of the metallic catalyst comprises oxidation number information of a copper in an oxidized form on the surface of the air purification unit.

12. The surface analysis system of claim 11, wherein the analysis unit is further configured to obtain the oxidation number information of the copper atom having an oxidation number of 0.

13. The surface analysis system of claim 12, wherein the control unit is configured to determine the relative presence of different oxidation states of the copper on the surface of the air purification unit.

14. The surface analysis system of claim 3, wherein the first light-source unit is further configured to continuously irradiate the inspection light onto the surface of the air purification unit as the air purification unit is operating to purify polluted air.

15. The surface analysis system of claim 14, wherein the analysis unit is further configured to determine the information regarding the surface of the air purification unit in real time as the air purification unit is operating to purify polluted air.

16. The surface analysis system of claim 15, wherein the polluted air comprises volatile organic compounds (VOCs) including at least one of gaseous formaldehyde, gaseous ammonia, gaseous acetaldehyde, gaseous acetic acid, and gaseous toluene.

17. The surface analysis system of claim 1, wherein the carrier comprises a porous ceramic support.

18. The surface analysis system of claim 11, wherein the carrier comprises a honeycomb structure.

19. An air purification device comprising the surface analysis system of claim 1.

20. The air purification device of claim 19, further comprising a pre-filter arranged on a side of the air purification unit.
